(19) United States
(12) Patent Application Publication
(10) Pub. No.: US 2022/0313500 A1
(43) Pub. Date: Oct. 6, 2022

(54) HYGIENIC ARTICLE WITH BACKSHEET ARRANGEMENT

(71) Applicant: Essity Hygiene and Health Aktiebolag, Göteborg (SE)

(72) Inventors: Verica TERENTIC, Göteborg (SE); Rikard Vilhelm MEYER NORÉN, Göteborg (SE); Mikael ANDERSSON, Göteborg (SE); Fred FREDERIX, Heijen (NL)

(73) Assignee: Essity Hygiene and Health Aktiebolag, Göteborg (SE)

(21) Appl. No.: 17/638,350

(22) PCT Filed: Aug. 28, 2019
(86) PCT No.: PCT/EP2019/072941

§ 371 Date: Feb. 25, 2022

(51) Int. Cl. A61F 13/49; A61F 13/496; A61F 13/15; A61F 13/42

(52) U.S. Cl. CPC A61F 13/49017; A61F 13/4902; A61F 13/496; A61F 13/15699; A61F 13/15756; A61F 13/42; A61F 2013/49025; A61F 2013/49092

(57) ABSTRACT

A hygienic article including a topsheet, a backsheet having first and second end edges extending in a lateral direction of the article and first and second side edges extending in a longitudinal direction of the article, and first and second leg elastic members extending along the first and second side edges of the backsheet. The backsheet includes first and second leg elastic anchoring portions extending along the side edges of the backsheet, the first and second leg elastic anchoring portions including two layers of backsheet material, with the first and second leg elastic members being arranged between the two layers of backsheet material, the first and second leg elastic members terminating at a distance from the first end edge of the backsheet, first leg elastic free portions are arranged at the first end edge of the hygienic article.

### TECHNICAL FIELD

The disclosure pertains to a hygienic article having first and second end edges and first and second side edges, the hygienic article comprising a topsheet, a backsheet arrangement having first and second end edges extending in a lateral direction of the article and first and second side edges extending in a longitudinal direction of the article, and leg elastic members extending along the first and second side edges of the backsheet arrangement.

A method for producing a hygienic article comprising a backsheet arrangement is also disclosed herein.

### BACKGROUND

Disposable hygienic articles, in particular disposable absorbent or non-absorbent articles which are worn in a pant-like fashion are commonly provided with elastic elements in selected areas, such as leg elastic arranged along leg edges of the hygienic article, and waist elastic arranged along the waist edge. Furthermore, the articles may be provided with side flaps or belt elements comprising fastener elements for closing the hygienic article when putting it on a user. Disposable articles having side flaps are usually produced by lengthwise production methods wherein the articles are continuously produced from continuous webs running in a machine direction which coincides with the length direction of the articles which are being produced. Continuous leg elastic elements are stretched and fed along with the traveling webs and are intermittently attached in their stretched state between a topsheet web and a backsheet web by means of adhesive or by ultrasonic welding or heat welding along the portions of the articles which are to form leg edges in the final hygienic article. Side flaps may be attached at a front end and/or at a rear end of each precursor hygienic article in the moving web of precursor hygienic articles and are attached between the topsheet web and the backsheet web by means of adhesive or by ultrasonic welding or heat welding.

It is generally desired that the leg elastic elements are present in the final hygienic article only along the leg edges and not in front and rear body portions of the hygienic article close to the front and rear waist edges. For this reason, the leg elastic elements are bonded to the topsheet web and the backsheet web only where functional leg elastic is desired. In a final production step when individual hygienic articles are cut off from the precursor web, the stretched leg elastic elements are simultaneously severed and will thereby snap back from the waist edges within the areas of the articles which are free from bonds between the leg elastic elements and the topsheet and backsheet webs. The snap-back portions of the leg elastic elements remain in the article as retracted, non-attached ends at the front and/or the rear portion of the hygienic article. However, it has been found that the application of leg elastic elements, in particular when the leg elastic involves snap-back portions, the provision of functional snap-back portions may negatively interfere with the incorporation of other elements such as side flaps, an absorbent core, waist elastic, etc.

Accordingly, there exists a need for an elasticated hygienic article having an improved construction allowing elements of the hygienic article to be more freely selected and adapted to functional requirements of the hygienic article.

### SUMMARY

Disposable hygienic articles having good functionality, and which may be provided with functional leg elastic independently of other components such as side flaps, an absorbent core, waist elastic, etc., may be achieved at least in part by the features of claim 1. A method for producing the disposable hygienic articles as disclosed herein is set out in claim 23.

Variations of the disclosure are set out in the dependent claims.

Disclosed herein is a hygienic article having first and second end edges and first and second side edges and comprising
a topsheet,
a backsheet arrangement having first and second end edges extending in a lateral direction of the article and first and second side edges extending in a longitudinal direction of the article, and
first and second leg elastic members extending along the first and second side edges,
   wherein the backsheet arrangement comprises first and second leg elastic anchoring portions extending along the side edges of the backsheet arrangement, the first and second leg elastic anchoring portions comprising two layers of backsheet material, with the first and second leg elastic members being arranged between the two layers of backsheet material, the leg elastic anchoring portions being fully or partly arranged laterally outward of the inner edge of the flap anchoring area, the first and second leg elastic members terminating at a distance from the first end edge of the backsheet arrangement, whereby first leg elastic free portions are arranged at the first end edge of the hygienic article.

The disposable hygienic article as disclosed herein may include an absorbent member which is disposed between the topsheet and the backsheet arrangement.

The disposable hygienic article as disclosed herein may be an absorbent or non-absorbent hygienic article such as a disposable article which is worn in a pant-like fashion. Accordingly, the first and second end edges of the hygienic article may be waist edges and first and second leg edges may be formed by the first and second side edges of the hygienic article.

Articles which are worn in a pant-like fashion include open open-type articles having fastener means such as fastener tabs or fastening belts for fastening of the article on a wearer, as well as pant-type articles which have closed side seams and which are put on a wearer in the same way as regular underpants. Pant-type articles may be provided with means for opening and reclosing the side seams. Hygienic articles which are worn in a pant-like fashion may be baby diapers or adult incontinence articles. Non-absorbent articles which are worn in a pant-like fashion may be swimwear, disposable underpants, covers for absorbent inserts, etc.

The hygienic articles as disclosed herein may have a rectangular shape, for ease of manufacturing. However, the side edges of the hygienic article may be cut to provide curved leg edges. The leg elastic elements may be arranged straight along the leg edges or may be arranged in a curve shape regardless of whether the hygienic article has a rectangular shape or is formed with curved leg edges.

The disposable hygienic article as disclosed herein may alternatively be an absorbent insert which is configured for being placed in a holder or an underpant and to be applied at least in the crotch portion of a wearer. Absorbent inserts include diapers, incontinence protectors and sanitary napkins.

The provision of a backsheet arrangement as disclosed herein provides freedom to place leg elastic elements in any selected part of the length of the hygienic article and at any suitable lateral position in the article, without having to take into consideration the placement and anchoring of waist elastic and side flaps such as fastening flaps or fastening belt parts. Furthermore, the width of the hygienic article between the side edges, as well as the width of an optional absorbent member may be freely selected to meet the functional requirements of the hygienic article with regard to fit and absorbency as the snap-back channels for the leg elastic elements may be arranged overlapping with the anchoring areas for the waist elastic and/or side flaps and not side-by-side, as in previous hygienic articles.

By means of the backsheet arrangement as disclosed herein, the need for arranging snap-back channels for leg elastic elements between the topsheet and the backsheet has been completely eliminated. In the hygienic articles as disclosed herein, all areas of the topsheet which are in direct contact with the backsheet arrangement may be bonded to each other such that no non-bonded areas having low tensile strength are created in the laminate which is formed by the topsheet and the backsheet arrangement. It may be preferred that the backsheet is attached to the topsheet in all overlapping portions at least outside the periphery of an absorbent member.

The disposable hygienic article as disclosed herein may comprise a first side flap extending from the first side edge of the hygienic article in the lateral direction of the hygienic article and a second side flap, such as first and second attachment flaps, extending from the second side edge in the lateral direction of the hygienic article. The first end edge of the hygienic article is a first waist edge and the second end edge of the hygienic article is a second waist edge, the first and second side flaps each having an inner end portion which is secured between the topsheet and the backsheet arrangement within first and second flap anchoring areas being arranged at the first waist edge of the hygienic article, each flap anchoring area having a lateral extension in the lateral direction of the hygienic article between an inner edge and an outer edge of the flap anchoring area and having a longitudinal extension in the longitudinal direction of the hygienic article between a waist edge of the side flap and a corresponding leg edge of the side flap.

The first and second leg elastic anchoring portions may be fully or partly arranged laterally outward of the inner edge of the flap anchoring area, the first and second leg elastic members terminating at a distance from the first waist edge, whereby first leg elastic free portions are arranged at the first waist edge.

In previously known hygienic articles, bond-free snap-back channels are arranged immediately inward of side flap anchoring areas in the lateral direction of the article. Such bond-free snap-back channels constitute weakened areas in the hygienic article which may tear when subjected to tensile forces applied perpendicular to the extension of the bond-free channels. The side flaps are used when putting on the hygienic article and may be in the form of fastening flaps or fastening belt parts. Fastening flaps are usually fastened onto a body portion of the hygienic article. If the fastening flaps are placed at a rear waist edge of the article, the fastening flaps are stretched out and brought forward and fastened on the front portion of the hygienic article. If the fastening flaps are placed at a front waist edge of the hygienic article, the fastening flaps are brought rearward and fastened on the rear portion of the hygienic article. In order to obtain a good fit of the hygienic article around the waist of the wearer, the fastening flaps are generally applied under tension to provide a snug fit around the waist of the wearer and to activate any laterally extending elastic elements, such as waist elastic, flap elastic, etc. The side flaps are commonly provided with fasteners such as mechanical fasteners or adhesive fasteners with a first fastener element placed on the side flap and a mating fastener element or fastener reception area placed on the body portion to which the side flaps are to be fastened. Fastening belt parts are stretched out similar to the fastening flaps and are usually fastened together at their outer ends. To this end, they may be provided with fastener elements, in analogy with the fastening flaps.

When the side flaps are pulled apart and tensioned during application of the hygienic article on a wearer, the weak non-bonded snap-back channels are exposed to tensile stress which may cause the material in the hygienic article to rupture along the snap-back channel.

The first end edge of the hygienic article may be a rear waist edge and the second end edge of the hygienic article may be a front waist edge. Hence, the first and second side flaps may be attachment flaps arranged at the rear waist edge of the hygienic article and being configured for fastening on the front portion of the hygienic article.

Alternatively, the first end edge of the hygienic article may be a front waist edge and the second end edge of the hygienic article may be a rear waist edge. With this configuration the first and second side flaps may be attachment flaps arranged at the rear waist edge of the hygienic article and being configured for fastening on the rear portion of the hygienic article.

A fastener may be arranged on least one attachment flap. It may be preferred that attachment flaps being provided with fasteners are arranged at the rear waist edge. When putting on the hygienic article on a wearer, the attachment flaps are stretched laterally and upwardly around the waist of the wearer. When both attachment flaps are provided with fasteners, the fasteners are generally fastened to a reception area on the outside of the article at the end of the article which is opposite to the attachment flaps. When only one attachment flap is provided with a fastener, the attachment flaps may be configured to be attached to each other in the manner of a belt.

The fasteners may be any type of fastener as known in the art, such as hook-type fasteners which are configured for being attached on a reception surface, such as on a surface having multiple loops. The reception surface may be an outer nonwoven layer of the backsheet arrangement or may be a particular loop material applied on an outer surface of the hygienic article. Alternatively the fasteners may be attached to attachment flaps arranged at the opposite end of the hygienic article.

The backsheet arrangement may comprise an inner layer and an outer layer. The inner and outer layers may be generally coextensive or the inner layer may have a greater width than the outer layer or vice versa.

The inner layer of the backsheet arrangement may be a barrier layer and the outer layer of the backsheet arrangement may be a nonwoven layer.

As set out herein, the outer layer may have a flat non-folded width in the lateral direction of the hygienic article which is greater than a flat non-folded width of the inner layer in the lateral direction of the hygienic article.

The leg elastic anchoring portions may be formed by folded-in edge portions of the backsheet arrangement with the leg elastic members placed within the folds. A configuration of this type may be advantageous, as the folds may form softer and more cushioning leg edges than a corresponding leg edge formed by cut edges of laminated layers.

The leg elastic anchoring portions may be formed by laminating a first sheet of backsheet material with a second sheet of backsheet material or by laminating a first sheet of backsheet material with first and second strips of backsheet material. Such configuration may be advantageous as it allows different materials to be selected for the outer and the inner surfaces of the elastic anchoring portions.

The backsheet arrangement may comprise a laminate of a polymeric film and a fibrous nonwoven sheet. The fibrous nonwoven sheet may provide a textile outer surface or outer cover of the hygienic article and/or a soft, body contacting surface along the leg elastic members. The polymeric film may provide liquid barrier properties. The polymeric film may be any suitable liquid barrier film, as known in the art, such as a breathable liquid barrier film.

A polymeric film layer may be laminated to a nonwoven layer or to nonwoven strips having edge folds in which elastic elements have been applied. The polymeric film may be applied such that it does not extend all the way to the side edges of a backsheet arrangement being defined by edge folds in a fibrous nonwoven layer.

Alternatively, a nonwoven layer may be folded in over a surface of a polymeric film layer to cover cut edges of the polymeric film layer.

The nonwoven sheet in the laminate may have a width in the lateral direction of the hygienic article which is greater than a width of the polymeric film in the lateral direction of the hygienic article. Hence, the nonwoven sheet may extend laterally beyond the polymeric film when in a planar state. The leg elastic anchoring portions may be formed completely by folds in the portions of the nonwoven sheet which extend outside the polymeric film. Such leg elastic anchoring portions are soft, pliable and highly breathable. Alternatively, the portions of the nonwoven sheet which extends laterally outside the polymeric film may be folded in over the polymeric film, so that the leg elastic anchoring portions comprise both the polymeric film and the nonwoven layer.

The first and second leg elastic anchoring portions may be formed solely from the outer layer.

As disclosed herein, the leg elastic anchoring portions may be formed solely by fibrous nonwoven material, either as a folded portion in a layer of nonwoven material or as a laminate of two separate layers of nonwoven material. Fibrous nonwoven elastic anchoring portions provide the leg elastic with good breathability, softness and cushioning ability.

The backsheet arrangement may be non-extensible in the lateral direction and/or in the longitudinal direction of the hygienic article. Alternatively, the backsheet arrangement may be extensible or elastically extensible in the lateral direction and/or in the longitudinal direction of the hygienic article. It may be desirable that the backsheet arrangement is non-extensible in the lateral direction of the hygienic article, as this may facilitate fitting of the hygienic article around the waist of a wearer. Furthermore, it may be advantageous to be able to use a non-extensible backsheet for cost-saving reasons.

The first and second leg elastic members may be any suitable leg elastic as known in the art, such as elastic bands, elastic strings, foam, etc. Each of the first and second leg elastic member may include multiple elastic strings or bands.

The leg elastic members may be attached in the leg elastic anchoring portions in a tensioned state. Alternatively, the first and second leg elastic members may be of a kind which can be activated e.g. by heat treatment or mechanical working. Such leg elastic members may be attached in a non-tensioned state and may be subsequently activated within a selected portion or portions of the leg elastic members.

The first leg elastic free portions may be formed by the leg elastic members being retracted from the first end edge. Retraction of the leg elastic members from the first leg elastic free portions may be accomplished by means of bond-free or substantially bond-free snap-back channels which are arranged in the longitudinal direction of the hygienic article, in the first leg elastic free portions of the backsheet arrangement.

After retraction, the leg elastic members have a length in the longitudinal direction of the hygienic article which is less than a length of the backsheet arrangement.

As disclosed herein, the first and second leg elastic members are shielded in the first and second leg elastic anchoring portions of the backsheet arrangement and are not in direct contact with the topsheet. Hence, the leg elastic elements are not directly bonded to the topsheet. Attachment of the first and second leg elastic members does not interfere with the attachment of side flaps or waist elastic members. This means that any snap-back portions of the leg elastic members may be placed wherever desired, such as in portions of the backsheet arrangement which overlap with side flap anchoring areas or with waist elastic areas, without negatively affecting secure anchoring of side flaps and/or waist elastic between the topsheet and the backsheet.

The two layers of backsheet material forming the leg elastic anchoring portions in the backsheet arrangement may be attached to each other with a first bonding pattern within the leg elastic anchoring portions and with a second bonding pattern within the leg elastic free portions, the first bonding pattern being an elastic anchoring pattern and the second bonding pattern being an elastic release pattern. The elastic anchoring pattern serves to firmly attach the leg elastic in a functional state, such as a stretched state in the backsheet arrangement. The elastic release pattern joins the layers of backsheet material to each other within the leg elastic free portions, but does not prohibit retraction of stretched leg elastic between the layers of backsheet material. Accordingly, the elastic release pattern allows severed stretched elastic to snap-back within the leg elastic free portion.

The first and second leg elastic members may terminate at a distance from the second end edge of the hygienic article, whereby second leg elastic free portions are arranged at the second end edge of the hygienic article.

The second leg elastic free portions may be formed by the first and second leg elastic members being retracted from the second end edge of the hygienic article.

The two layers of backsheet material forming the leg elastic anchoring portions may be attached to each other by means of adhesive or by means of ultrasonic welding or by means of a combination of adhesive and ultrasonic welding.

The outer layer of the backsheet arrangement may include a spunbond material and optionally a meltblown material.

The hygienic article as disclosed herein may be a diaper comprising side panels which are attached to the backsheet arrangement along the side edges of the hygienic article at the first end edge and/or at the second end edge of the hygienic article. As an alternative, the hygienic article may be a panel-type diaper, a front end edge of the backsheet arrangement being joined to a front body panel and a rear end edge of the backsheet arrangement being joined to a rear body panel.

As disclosed herein, the article may include fastening elements for fastening the article in a pant-like configuration on a wearer.

In a hygienic article as disclosed herein a third side flap may extend from the first side edge in the lateral direction of the article and a fourth side flap may extend from the second side edge in the lateral direction of the article, the third and fourth side flaps being arranged at the second end edge. The third and fourth side flaps may be attachment flaps being configured to cooperate with first and second attachment flaps which are arranged at the first end edge.

The third and fourth side flaps may each have an inner end portion which is firmly attached between the topsheet and the backsheet arrangement within third and fourth flap anchoring areas, each flap anchoring area having a lateral extension in the lateral direction of the hygienic article between an inner edge and an outer edge of the flap anchoring area and having a longitudinal extension in the longitudinal direction of the hygienic article between a waist edge of the side flap and a corresponding leg edge of the side flap.

The leg elastic anchoring portions may be fully or partly arranged laterally outward of the corresponding inner edges of the third and fourth flap anchoring areas.

The hygienic articles as disclosed herein may be produced by a method including the steps of:
producing a continuous backsheet web by continuously feeding first and second leg elastic members between first and second layers of backsheet material and securing the first and second leg elastic members between the first and second layers of backsheet material in first and second leg elastic anchoring portions arranged intermittently along corresponding first and second longitudinal edges of the continuous backsheet web and being separated by leg elastic release portions, the first and second leg elastic members being secured to the continuous backsheet web in the leg elastic anchoring portions while being held in a stretched state;
forming a continuous article web of interconnected hygienic articles, the continuous article web incorporating the continuous backsheet web;
severing individual hygienic articles from the continuous article web and simultaneously severing the first and second leg elastic members in the leg elastic release portions between the intermittently arranged leg elastic anchoring portions, causing the severed leg elastic elements to relax and retract within the leg elastic release portions.

In the method as disclosed herein, forming the continuous article web may include joining one or more of the following components to the continuous backsheet web:
a topsheet;
an absorbent member;
a waistband component;
side panels;
an acquisition web;
fastener elements;
barrier cuffs.

### BRIEF DESCRIPTION OF THE DRAWINGS

The hygienic articles as disclosed herein will be further explained hereinafter with reference to the appended drawings wherein:

FIG. 1 shows a planar view of a hygienic article in the form of a diaper, from the side which is intended to be facing a wearer during use of the diaper;

FIG. 2 shows a cross-section through the diaper in FIG. 1, taken along the line II-II in FIG. 1;

FIG. 3 shows a corner portion of the hygienic article in FIG. 1 as indicated at Ill in FIG. 1;

FIG. 4 shows the hygienic article in FIG. 1 during application to a wearer;

FIGS. 5a-5j show cross-sections of different backsheet arrangements; and

FIG. 6 shows a detail of a backsheet arrangement comprising an elastic anchoring portion and an elastic free portion with different bonding patterns; and

FIG. 7 shows a schematic representation of a method as disclosed herein.

### DETAILED DESCRIPTION

Different aspects of the present disclosure will be described more fully hereinafter with reference to the enclosed drawings. The embodiments disclosed herein can, however, be realized in many different forms and should not be construed as being limited to the aspects set forth herein.

It is to be understood that the drawings are schematic and that individual components, such as layers of material are not necessarily drawn to scale.

With initial reference to FIGS. 1, 2, 3 and 4, there is shown a hygienic article 1 in the form of a diaper. As set out herein, the backsheet arrangement as disclosed herein may equally well be applied to any type of externally worn hygienic article, such as self-containing garments or inserts which are worn together with an outer panty or holder.

The hygienic article 1 has a first end edge 2 and a second end edge 4 and first and second side edges 6, 8. The first end edge 2 of the hygienic article 1 which is shown in FIG. 1 is a rear waist edge and the second end edge 4 of the hygienic article 1 is a front waist edge. The first and second end edges 2, 4 extend in a lateral direction T of the hygienic article 1 and the side edges 6,8 extend in a longitudinal direction L of the hygienic article 1.

The hygienic article 1 which is shown in FIGS. 1-4 comprises a topsheet 9 and a backsheet arrangement 10. An absorbent member 11 is disposed between the topsheet 9 and the backsheet arrangement 10. The hygienic article 1 may be divided in the longitudinal direction into a front portion 3, a rear portion 5 and a crotch portion 7 located between the front portion 3 and the rear portion 5.

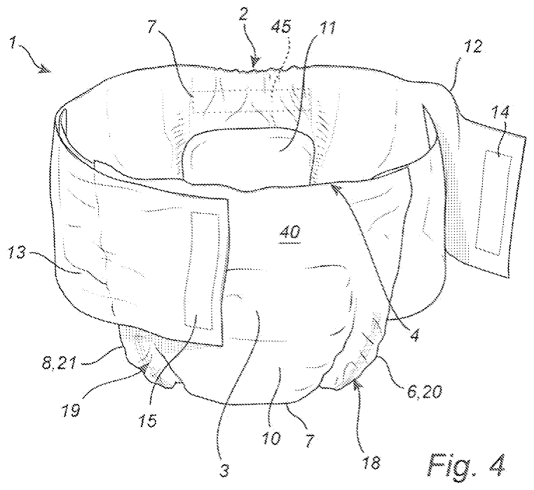

A first side flap 12 is arranged extending from the first side edge 6 in the lateral direction T of the hygienic article 1 and a second side flap 13 extends from the second side edge 8 in the lateral direction of the hygienic article. The first and second side flaps 12, 13 are attachment flaps and are provided with fasteners 14, 15 for securing the hygienic article 1 in a pant-like configuration as shown in FIG. 4.

First and second leg elastic members 18, 19 extend in the longitudinal direction L of the hygienic article along first and second side edges 20, 21 of the backsheet arrangement 10. In the hygienic article 1 which is shown in FIGS. 1-4, the side edges of the backsheet arrangement 20, 21 coincide with the side edges 6, 8 of the hygienic article 1 and the end edges 2,4 of the backsheet arrangement 10 coincide with the end edges 2,4 of the hygienic article 1. It is not a prerequisite of the hygienic articles as disclosed herein that the end and side edges of the backsheet arrangement coincide with the end and side edges of the hygienic article.

The first and second side flaps 12, 13 each has an inner end portion 12′, 13′ which is secured between the topsheet 9 and the backsheet arrangement 10 within first and second flap anchoring areas 22, 23. The first and second flap anchoring areas 22, 23 are arranged at the first end edge 2. Each flap anchoring area 22, 23 has a lateral extension in the lateral direction T of the hygienic article 1 between an inner edge 25 and an outer edge 26 of the flap anchoring area 22, 23 and has a longitudinal extension in the longitudinal direction L of the hygienic article 1 between a waist edge 27 of the side flap 12, 13 and a corresponding leg edge 28 of the side flap 12, 13. In the hygienic article 1 which is shown in FIGS. 1-4, the waist edges 27 of the first and second side flaps 12, 13 coincide with the first end edge 2, i.e. the rear waist edge of the hygienic article 1.

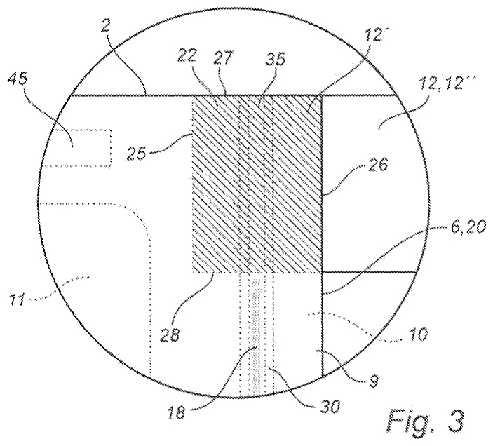

The backsheet arrangement 10 comprises first and second leg elastic anchoring portions 30, 31 extending along the side edges 6, 8 of the hygienic article. The first and second leg elastic anchoring portions 30, 31 each comprise two layers of backsheet material 32, 33, the first and second leg elastic members 18, 19 being arranged between the two layers of backsheet material 32, 33. In FIGS. 1 and 3, the leg elastic anchoring portions 30, 31 and the leg elastic members 18, 19 are shown arranged laterally completely outward of the inner edges 25 of the flap anchoring areas 22, 23. The first and second leg elastic members 18, 19 terminate at a distance from the first end edge 2, whereby first leg elastic free portions 35, 36 are arranged at the first end edge 2. The first leg elastic free portions 35, 36 are arranged between the two layers of backsheet material 32, 33 and are placed at the ends of the leg elastic members 18, 19 within the areas of the first and second flap anchoring areas 22, 23.

The first and second leg elastic members 18, 19 are attached in the first and second leg elastic anchoring portions 30, 31 in a tensioned state. During production of the hygienic article 1, the leg elastic members 18, 19 have been bonded to the backsheet arrangement 10 such that they stay bonded within the leg elastic anchoring portions 30, 31 also after severing of the leg elastic members 18, 19. The portions of the leg elastic members 18, 19 which during production extend outside the leg elastic anchoring portions 30, 31 remain non-bonded or only weakly bonded during production and will snap-back from the first end edge 2 when the hygienic article 1 is finally severed from a production web. Accordingly, the first leg elastic free portions 35, 36 are formed by means of snap-back channels which are formed at the end edges 2, 4 of the hygienic article 1.

After retraction, the first and second leg elastic members 18, 19 have a length in the longitudinal direction L of the hygienic article 1 which is less than a total length of the hygienic article 1.

As disclosed herein, the leg elastic members 18, 19 are shielded in the leg elastic anchoring portions 30, 31 of the backsheet arrangement and are not in direct contact with the topsheet 9. Hence, the leg elastic members 18, 19 are not directly bonded to the topsheet 9. Attachment of the leg elastic members 18, 19 is made in a different plane from the attachment plane between the topsheet 9 and the backsheet arrangement 10 and does not interfere with the attachment of the side flaps 12, 13 as the bond-free or substantially bond-free snap-back channels provided in the elastic free areas are arranged in a different plane from the plane of the first and second flap anchoring areas 22, 23. In a hygienic article as disclosed herein, the need for snap-back channels between the topsheet and the backsheet has been completely eliminated. In the hygienic articles as disclosed herein, all areas of the topsheet which are in direct contact with the backsheet arrangement may be bonded to each other such that no weakened areas are created in the laminate which is formed by the topsheet and the backsheet arrangement. It may be preferred that the backsheet is attached to the topsheet in all overlapping portions at least outside the periphery of the absorbent member. Preferably, there is no bond-free area between the end edges of the absorbent member and the waist edges of the hygienic article. Furthermore, there are no bond-free channels which may constitute weakened portions acting as tear indications when putting on the hygienic article.

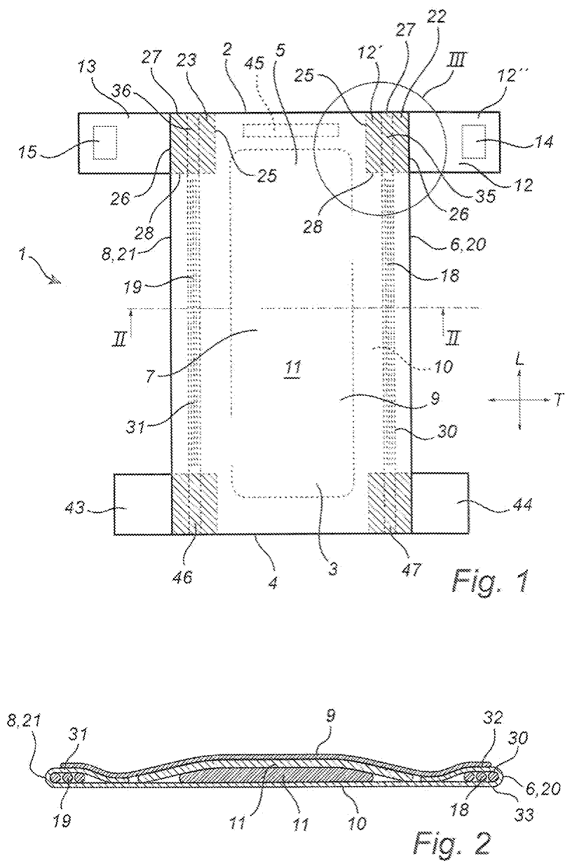

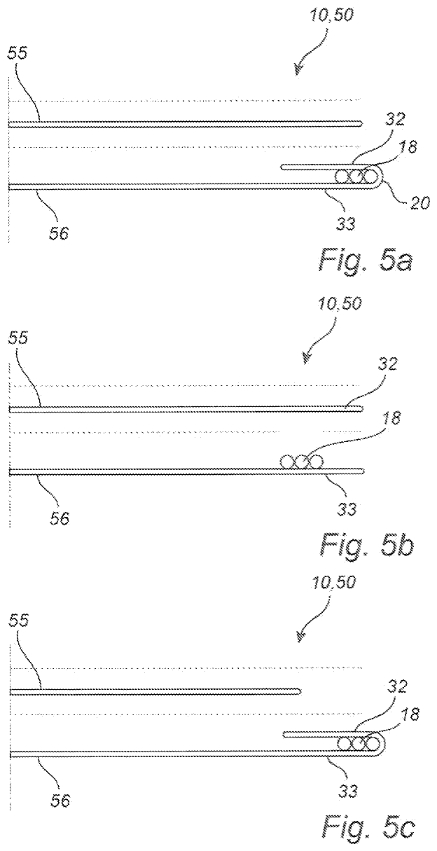

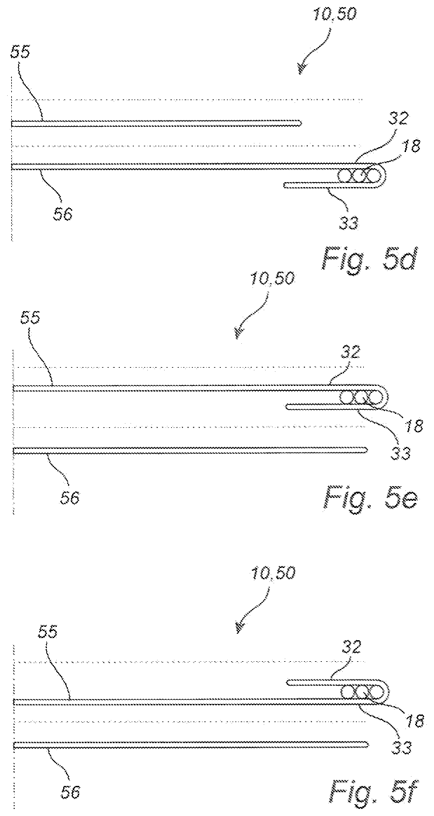

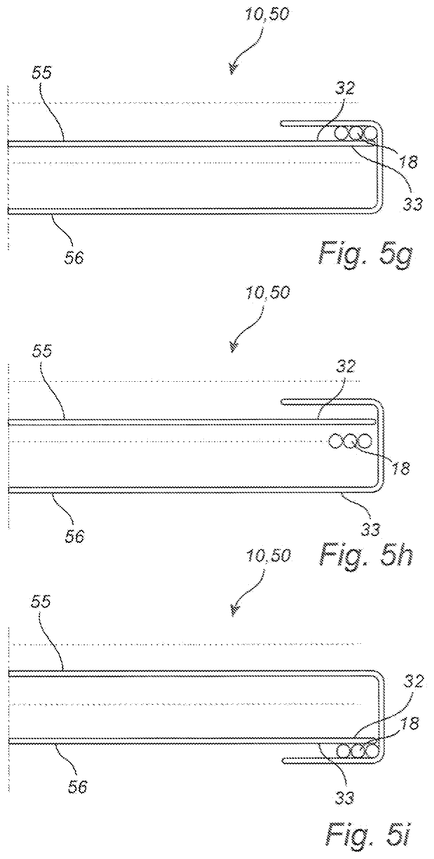

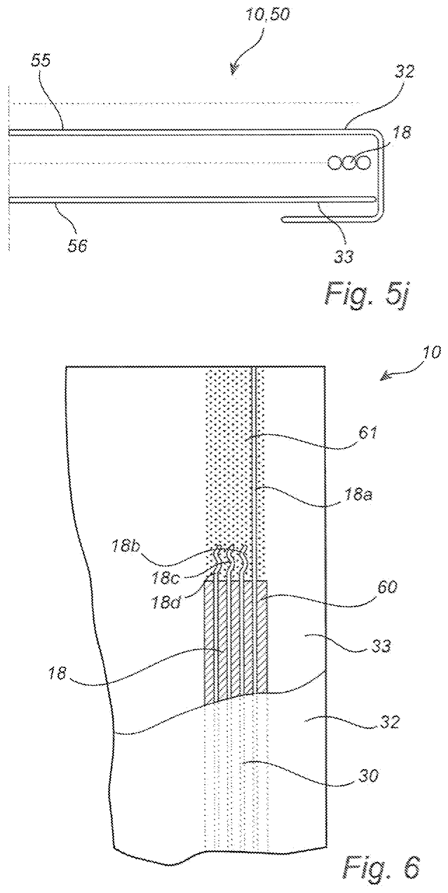

In the hygienic article 1 which is shown in FIGS. 1-4, the leg elastic anchoring portions 30, 31 are formed by folded-in edge portions of the backsheet arrangement 10, as is seen in FIG. 2. The leg elastic anchoring portions may be formed in the backsheet arrangement 10 in any other way as disclosed herein as long as the leg elastic elements are attached between two layers of the backsheet arrangement. Some alternative configurations of the leg elastic anchoring portions are those shown in FIGS. 5a-5j.

The first and second side flaps 12, 13 are attachment flaps which are tensioned when putting on the hygienic article and need to be firmly anchored between the topsheet 9 and the backsheet 10 to ascertain that they do not come off the article when exposed to laterally applied pulling forces. The first and second side flaps 12, 13 may be made from any suitable material as known in the art, such as fibrous nonwoven material. The first and second side flaps 12, 13 may be elastic or may be elastically stretchable, at least in the lateral direction of the hygienic article 1. The first and second side flaps 12, 13 may be breathable and are preferably soft and pliable for providing good body conformance and comfort.

The first and second side flaps 12, 13 which are shown in FIGS. 1-4 are provided with fastener elements 14, 15 which may be of any suitable kind such as adhesive or mechanical fasteners. A common type of mechanical fastener is hook-and-loop fasteners. In the hygienic article 1 in FIGS. 1-4, the fastener elements 14, 15 may be seen as hook-type fasteners, i.e. fasteners having multiple protruding elements which may engage with a corresponding loop-type fastener such as a loop-type nonwoven material. The receiving area 40 is shown in FIG. 4 to be located on the outside of the front portion 3 of the hygienic article 1, which in this case is an outer nonwoven layer of the backsheet arrangement 10.

Alternatively, the fastener elements 14, 15 may be seen as adhesive fastener tapes which are arranged to be attached on a receiving area arranged on the front portion 3 of the hygienic article 1. As disclosed herein, the first and second side flaps 12, 13 may alternatively be arranged to be attached to third and fourth side flaps arranged at the opposite waist edge. Accordingly, the first and second side flaps 12, 13 which are arranged at the rear waist edge 2, may be configured to be attached to the third and fourth side flaps 43, 44 which are located at the front waist edge 4. Furthermore, the first and second side flaps 12, 13 may have a lateral extension which is large enough such that the first and second side flaps 12, 13 can be fastened to each other in the manner of a belt. It is to be understood that in a hook-and-loop type fastener arrangement, the hook-type fastener elements may be arranged on a receiving surface while the loop type fastener elements are arranged on a first and/or a second side flap.

The provision of a backsheet arrangement 10 as disclosed herein makes it possible to provide selected leg elastification while avoiding having snap-back channels formed by bond-free areas between the topsheet and the backsheet. Such bond-free snap-back channels constitute weakened areas in the hygienic article 1 which may tear when subjected to tensile forces applied perpendicular to the extension of the bond-free channels as the first and second side flaps 12, 13 are stretched out and brought forward and fastened on the front portion 3 of the hygienic article 1 under tension as illustrated in FIG. 4.

In previously known hygienic articles, bond-free snap-back channels are arranged immediately inward of side flap anchoring areas in the lateral direction of the hygienic article in order to avoid interference with the fastening of the side flaps. When the prior art side flaps are pulled apart and tensioned during application of the hygienic article on a wearer, the weak non-bonded snap-back channels are exposed to tensile stress which may cause the material in the hygienic article to rupture along the snap-back channel.

The third and fourth side flaps 43, 44 as shown in the figures are optional to the hygienic articles as disclosed herein. The third and fourth side flaps 43, 44, may be made from generally the same materials as the first and second side flaps 12, 13. The third and fourth side flaps 43, 44 may be provided with fastener elements, and may be arranged to contribute to fastening of the hygienic article on a wearer. In a hygienic article such as the hygienic article 1 in FIGS. 1-4 where the third and fourth side flaps 43, 44 are not fastening flaps, they will generally not be exposed to lateral tension and need not be as firmly anchored to the hygienic article as is the case for side flaps which are involved in tensioned fastening of the hygienic article around the waist of a wearer. Hence, in alternative to being attached between the topsheet 9 and the backsheet arrangement 10 in the same manner as the first and second side flaps 12,13, the third and fourth side flaps 43, 44 may be attached on the outer, wearer-facing surface of the topsheet 9 or on the outer, garment-facing surface of the backsheet arrangement 10.

When the third and fourth side flaps 43, 44 are attached between the topsheet 9 and the backsheet arrangement 10 in the same manner as the first and second side flaps 12,13, the leg elastic members 18, 19 are preferably applied in the same manner as in the rear portion 5 of the hygienic article 1, i.e. with elastic free portions 46, 47 created by arranging snap-back channels in the backsheet arrangement 10 at the front waist edge 4. The snap-back channels are arranged between the two layers of backsheet material 32, 33 between which the leg elastic members 18, 19 are applied.

The hygienic article 1 in FIGS. 1-4 is further shown to include an optional waist elastic member 45. The waist elastic member 45 is located at the rear waist edge 2 and extends in the lateral direction along only a part of the rear waist edge 2. Any other type of waist elastic may be used, as known in the art.

The absorbent member 11 is disposed between the topsheet 9 and the backsheet arrangement 10 to absorb liquid, such as urine or other bodily fluids, which has passed through the topsheet 9. The absorbent member 11 may be a single-layer structure or may be a layered structure as shown in FIG. 2. The absorbent member 11 may comprise suitable amounts of superabsorbent material. Such superabsorbent material is well known in the field of hygienic articles, and is constituted by water-swellable and water-insoluble material which is capable of absorbing large quantities of fluid upon formation of a hydrogel. The absorbent component may contain superabsorbent material in the form of fibers or particles of absorbent polymer material. For example, the superabsorbent material may be surface cross-linked, partially neutralized polyacrylates. The superabsorbent material, e.g., the superabsorbent fibers or particles, may be mixed with other absorbent or liquid uptake material or materials, such as cellulose fluff pulp, and/or be arranged in pockets or layers in the absorbent member 11.

The absorbent member 11 may further comprise components for improving properties of the absorbent member 11, such as integrity and strength. For example, the absorbent member 11 may comprise a binder or binders, such as binder fibers. Resilient fibers, chemically stiffened fibers, etc. may be present in the absorbent member 11 to counteract wet-collapse of cellulosic fibers. Such fibers may also be useful in retaining a fluid transporting capillary network in the absorbent component so that absorbent fluid may be distributed in the absorbent component and be absorbed by superabsorbent material also in parts of the absorbent component outside the initial wetting area of the hygienic article.

With reference to FIG. 5, there is shown a number of cross-sections through a leg elastic snap-back portion 50 of different backsheet arrangements 10 which may be used in any hygienic article as disclosed herein. The snap-back portions are shown as they appear during production of a hygienic article as disclosed herein, before severing of individual articles from the production web of interconnected articles, i.e. before the tensioned elastic members 18 have been cut off and retracted from the snap-back portion 50 to create a leg elastic free portion. The broken lines in all of FIGS. 5a-5j indicate the presence of construction bonding such as construction adhesive, thermo-bonding or ultrasonic bonding. Construction bonding is used to bond the layers and components of the backsheet arrangements 10 together and to bond the backsheet arrangement to other components of the hygienic article such as a topsheet, side flaps, etc. The leg elastic anchoring portions of the backsheet arrangements in FIGS. 5a-5j have the same appearance as the snap-back portions 50 shown in FIGS. 5a-5j, except that construction bonding is present to anchor the leg elastic members 18 in the leg elastic anchoring portions.

All the backsheet arrangements 10 which are shown in FIGS. 5a-5j comprise a first sheet material 55 and a second sheet material 56. The backsheet arrangements are intended to be incorporated into a hygienic article as disclosed herein with the first sheet material 55 being an inner layer facing toward the topsheet of the hygienic article and with the second sheet material 56 being an outer layer facing away from the topsheet of the hygienic article. The first sheet material 55 may be a liquid barrier material such as a liquid-impermeable plastic film, a nonwoven sheet which has been coated with a liquid barrier material, or some other flexible material sheet which has the ability to withstand liquid penetration. It may be advantageous if the liquid-impervious barrier is breathable, i.e. permits the passage of water vapour through the barrier sheet. The first sheet material 55 may be stretchable, elastically stretchable or non-elastic. The second sheet material 56, may be an outer covering sheet which may have a textile or textile-like appearance and/or at textile or textile-like feel. The second sheet material 56 may be a plastic film material, e.g. an embossed, reduced gloss film or a fibrous nonwoven material. Fibrous nonwoven materials have an excellent textile appearance and feel and low gloss and may be preferred as outer cover layers. However, it is to be understood that a backsheet arrangement as disclosed herein may consist of a single sheet material having the elastic elements arranged in side folds in the sheet material. Such backsheet arrangements would be accomplished e.g. by omitting the first sheet material 55 in FIGS. 5a, 5c and 5d, and by omitting the second sheet material 56 in FIGS. 5e and 5f.

Furthermore, although the first and second sheets of material are shown as continuous sheets, one of the first and second sheet materials may be a strips of backsheet material with a corresponding strip of backsheet material used in the leg elastic anchoring portion which is arranged along the opposite side edge of the backsheet arrangement.

In the backsheet arrangement which is shown in FIG. 5a, the elastic members 18 are arranged in an edge fold in the second sheet material 56, the second sheet material 56 forming both a first layer of backsheet material 32 and a second layer of backsheet material 33 between which two layers 32, 33 of backsheet material the leg elastic members 18 are applied. The edge-fold in the second sheet material 56 is made such that the folded-in edge portion 32 of the second sheet material 56 is located between the first sheet material 55 and the second sheet material 56. The first sheet material 55 is attached to the second sheet material 56 and is almost coextensive with the second sheet material in that the first sheet material 55 terminates in the lateral direction only a small distance from side edge 20 of the backsheet arrangement, the side edge 20 being defined by the edge fold in the second sheet material 56. In this manner, the first sheet material 55 extends in over the leg elastic members 18.

In the backsheet arrangement which is shown in FIG. 5b, the first sheet material 55 and the second sheet material 56 are non-folded and generally coextensive and form the first and second layers of backsheet material 32, 33 between which the leg elastic members 18 are arranged.

The backsheet arrangement which is shown in FIG. 5c differs from the backsheet arrangement 10 which is shown in FIG. 5a in that the first sheet material 55 extends laterally only slightly in over the folded-in edge portion of the second sheet material 56. Hence, the first sheet material 55 does not extend in over the elastic elements.

The backsheet arrangement which is shown in FIG. 5d differs from the backsheet arrangement 10 which is shown in FIG. 5c in that the edge-fold in the second sheet material 56 is made such that the folded-in edge portion of the second sheet material 56 is located on the side of the second sheet material 56 which faces away from the first sheet material 55.

In the backsheet arrangement which is shown in FIG. 5e, the leg elastic members 18 are arranged in an edge fold in the first sheet material 55, the first sheet material 55 forming both a first layer of backsheet material 32 and a second layer of backsheet material 33 between which two layers of backsheet material 32, 33 the leg elastic members 18 are applied. The edge-fold in the first sheet material 55 is made such that the folded-in edge portion 33 of the first sheet material 55 is located between the first sheet material 55 and the second sheet material 56. The second sheet material 56 is attached to the first sheet material 55 and is almost coextensive with the first sheet material in that the second sheet material 56 terminates in the lateral direction only a small distance from the side edge 20 of the backsheet arrangement 10, the side edge 20 being defined by the edge fold in the first sheet material 55. In this manner, the second sheet material 56 extends in over the leg elastic members 18.

The backsheet arrangement which is shown in FIG. 5f differs from the backsheet arrangement 10 which is shown in FIG. 5e in that the edge-fold in the first sheet material 55 is made such that the folded-in edge portion 32 of the first sheet material 55 is located on the side of the first sheet material 55 which faces away from the second sheet material 56.

In the backsheet arrangement which is shown in FIG. 5g, the leg elastic members 18 are arranged along a non-folded edge portion 33 of the first sheet material 55 on the surface of the first sheet material 55 which faces away from the second sheet material 56 and are covered by a folded-in edge portion 32 of the second sheet material 56, the folded-in edge portion 32 being folded onto the surface of the first sheet material 55 which faces away from the second sheet material 56.

The backsheet arrangement which is shown in FIG. 5h, differs from the backsheet arrangement which is shown in FIG. 5g in that the leg elastic members 18 are arranged on the surface of the first sheet material 55 which faces towards the second sheet material 56.

The backsheet arrangements which are shown in FIGS. 5i and 5j, are upside-down variants of the backsheet arrangements which are shown in FIGS. 5g and 5h. Accordingly, in FIG. 5i the leg elastic members 18 are arranged on the surface of the second sheet material 56 which faces away from the first sheet material 55 and are covered by a folded-over portion 33 of the first sheet material 55. In FIG. 5j, the leg elastic members 18 are arranged between the first and second sheet materials 55, 56.

With reference to FIG. 6, the two layers of backsheet material 32, 33 forming a leg elastic anchoring portion 30 may be attached to each other with a first bonding pattern 60 within the leg elastic anchoring portion 30 and with a second bonding pattern 61 within the leg elastic free portion 35. The first bonding pattern 60 is an elastic anchoring pattern and the second bonding pattern 61 is an elastic release pattern.

The elastic anchoring pattern 60 serves to firmly attach the elastic elements 18a-d of the leg elastic member 18 in a stretched functional state in the backsheet arrangement 10. The elastic release pattern 61 joins the layers of backsheet material 32, 33 to each other within the leg elastic free portions 35, but does not prohibit retraction of tensioned leg elastic between the layers of backsheet material. Accordingly, the elastic release pattern allows severed stretched elastic to snap-back within the leg elastic free portion.

The two layers of backsheet material 32, 33 forming the leg elastic anchoring portion 30 and the elastic release portion 35 may be attached to each other by means of adhesive. The adhesive is then applied with a greater surface coverage and/or which a greater basis weight within the area of the elastic anchoring pattern 60 than within the area of the elastic release pattern 61. Bonding may alternatively be effected by means of heat welding or ultrasonic welding as set out in EP 3 056 176 A1 and US 2016/0058624 A1. A denser boding pattern is then created in the leg elastic anchoring portion 30 than in the elastic release portion 35. The sparsely dispersed bond points or the adhesive in the elastic release pattern 61 are insufficient to counteract the retractive forces acting on the tensioned elastic elements 18a-d when the elastic elements 18a-d are severed. Consequently, the severed elastic elements 18b-d are released from any bonds in the elastic release pattern 61 and snap back from the position illustrated by the elastic element 18a, towards the leg elastic anchoring portion 30, as illustrated by the elastic elements 18b-18d.

Bonding between the first sheet material 55 and the second sheet material 56 outside the elastic anchoring portion 30 and the elastic release portion 35 may be made with any suitable bond pattern and bonding method.

The elastic member 18 in FIG. 6 is shown as a multistring elastic member with four elastic elements 18a-18e. it is to be understood that any suitable number of elastic elements may be used, such as 1-30 elastic elements, 2-20 elastic elements or 5 to 15 elastic elements.

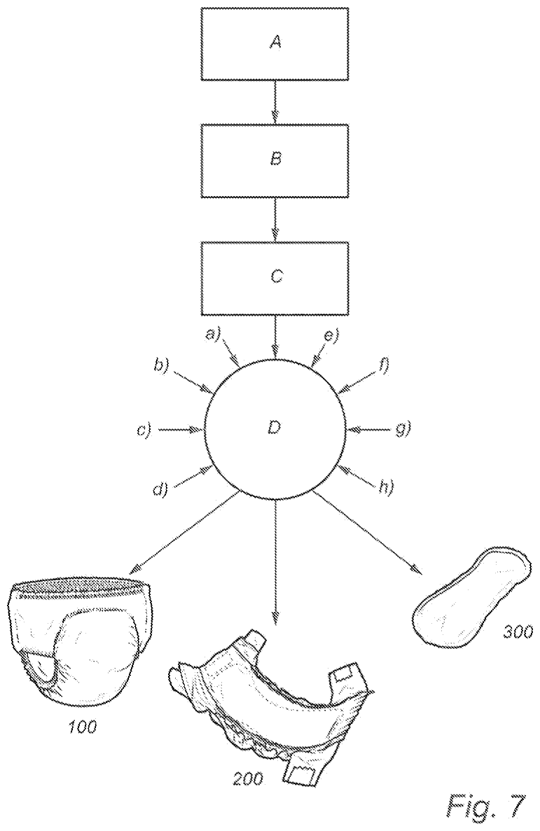

With reference to FIG. 7, a method for producing a hygienic article as disclosed herein includes the following steps and procedures:

A—continuously feeding a first layer of backsheet material, the first layer of backsheet material being a continuous web having first and second side edges. The first layer of backsheet material may be an outer or inner backsheet layer in the hygienic article which is being produced, as set out herein.
B—continuously applying first and second leg elastic members along the first and second side edges of the first layer of backsheet material.
C—intermittently securing the first and second leg elastic members between the first layer of backsheet material and a second layer of backsheet material. The first and second leg elastic members are secured in first and second leg elastic anchoring portions which are arranged intermittently along the first and second longitudinal edges of the continuous first layer of backsheet material and which are separated by leg elastic release portions. The first and second leg elastic members are secured between the first and second layers of backsheet material while being held in a stretched state.

The second layer of backsheet material may be a second web of material which is continuous both in the machine direction and in the cross machine direction and has a width in the cross machine direction which is sufficient to cover the first and second leg elastic members. Alternatively, as set out herein, the second layer of backsheet material covering the first and second leg elastic members may be first and second continuous bands of material which are applied only along the first and second side edges of the first layer of backsheet material. A further alternative, as set out herein, is that the second layer of backsheet material is provided by folding-in edge portions of the first layer of backsheet material over the first and second leg elastic members.

D—obtaining a continuous backsheet web having leg elastic members arranged therein and being shielded from direct contact with other components of a hygienic article by being applied between two layer of backsheet material.

The backsheet web which is obtained at D may be further processed into a continuous article web of interconnected hygienic articles by joining one or more additional components to the continuous backsheet web such as:

a) a topsheet;
b) an absorbent member;
c) a waistband component;
d) side panels;
e) an acquisition web;
f) fastener elements;
g) barrier cuffs;
h) a wetness indicator.

The further processing of the backsheet web obtained at D may be performed directly on the moving continuous backsheet web. Alternatively, the backsheet web may be made as an intermediate article which is rolled up and converted into hygienic articles at a later time and/or at another location.

When all additional components have been added to the continuous backsheet web, individual hygienic articles are severed from the continuous article web while simultaneously severing the first and second leg elastic members in the leg elastic release portions between the intermittently arranged leg elastic anchoring portions, causing the severed leg elastic elements to relax and retract within the leg elastic release portions.

The articles 100, 200 and 300 which are shown in FIG. 7 illustrate that different types of hygienic articles may be produced using the continuous backsheet web. The type of hygienic article which is produced is determined by the additional components which are added to the continuous backsheet web, and on additional process steps which are performed, such as forming side seams, applying printed or embossed patterns, etc. The individual articles 100, 200, 300 may finally be folded and packaged, as known in the art.

1. A hygienic article having first and second end edges and first and second side edges and comprising a topsheet,
a backsheet arrangement having first and second end edges extending in a lateral direction of the article and first and second side edges extending in a longitudinal direction of the article,
first and second side flaps extending laterally from the first and second side edges of the hygienic article, respectively, and
first and second leg elastic members extending along the first and second side edges of the backsheet arrangement,
wherein the backsheet arrangement comprises first and second leg elastic anchoring portions extending along the side edges of the backsheet arrangement, the first and second leg elastic anchoring portions comprising two layers of backsheet material, with the first and second leg elastic members being arranged between the two layers of backsheet material, the first and second leg elastic members terminating at a distance from the first end edge of the backsheet arrangement, whereby first leg elastic free portions are arranged at the first end edge of the hygienic article.

2. The hygienic article according to claim 1, wherein an absorbent member is disposed between the topsheet and the backsheet arrangement.

3. The hygienic article according to claim 1, wherein the first and second end edges of the hygienic article are waist edges and wherein first and second leg edges are formed by the first and second side edges of the hygienic article.

4. The hygienic article according to claim 1, wherein the backsheet arrangement comprises an inner layer and an outer layer.

5. The hygienic article according to claim 4, wherein the inner layer of the backsheet arrangement is a barrier layer and the outer layer of the backsheet arrangement is a nonwoven layer.

6. The hygienic article according to claim 4, wherein the outer layer has a flat non-folded width in the lateral direction of the hygienic article which is greater than a flat non-folded width of the inner layer in the lateral direction of the hygienic article.

7. The hygienic article according to claim 1, wherein the backsheet arrangement is non-extensible in the lateral direction of the hygienic article.

8. The hygienic article according to claim 4, wherein the first and second leg elastic anchoring portions are formed solely from the outer layer.

9. The hygienic article according to claim 1, wherein the elastic members include multiple elastic strings.

10. The hygienic article according to claim 1, wherein the first and second elastic members are attached inside the leg elastic anchoring portions in a tensioned state.

11. The hygienic article according to claim 9, wherein the first leg elastic free portions are formed by the first and second leg elastic members being retracted from the first end edge.

12. The hygienic article according to claim 1, wherein the two layers of backsheet material forming the leg elastic anchoring portions are attached to each other with a first bonding pattern within the leg elastic anchoring portions and with a second bonding pattern within the leg elastic free portions, the first bonding pattern being an elastic anchoring pattern and the second bonding pattern being an elastic release pattern.

13. The hygienic article according to claim 1, wherein the first and second leg elastic members terminate at a distance from the second end edge of the backsheet arrangement, whereby second leg elastic free portions are arranged at the second end edge of the backsheet arrangement.

14. The hygienic article according to claim 13, wherein the second leg elastic free portions are formed by the first and second leg elastic members being retracted from the second end edge.

15. The hygienic article according to claim 1, wherein the two layers of backsheet material forming the leg elastic anchoring portions are attached to each other by means of adhesive.

16. The hygienic article according to claim 1, wherein the two layers of backsheet material forming the leg elastic anchoring portions are attached to each other by means of ultrasonic welding.

17. The hygienic article according to claim 4, wherein the outer layer of the backsheet arrangement includes a spunbond material and optionally a meltblown material.

18. The hygienic article according to claim 1, wherein the leg elastic anchoring portions 4 are formed by folded-in edge portions of the backsheet arrangement.

19. The hygienic article according to claim 1, wherein the leg elastic anchoring portions are formed by laminating a first sheet of backsheet material with a second sheet of backsheet material or by laminating a first sheet of backsheet material with first and second strips of backsheet material.

20. The hygienic article according to claim 4, wherein the outer layer and/or the inner layer of the backsheet arrangement comprises a breathable polymeric film.

21. The hygienic article according to claim 1, wherein the side panels are attached to the backsheet arrangement along the side edges of the hygienic article at the first end edge and/or at the second end edge of the hygienic article or wherein the hygienic article is a panel-type diaper, a front end edge of the backsheet arrangement being joined to a front body panel and a rear end edge of the backsheet arrangement being joined to a rear body panel.

22. The hygienic article according to claim 1, wherein the hygienic article comprises fastening elements for fastening the article in a pant-like configuration on a wearer.

23. A method for producing a hygienic article the method including the steps of:
producing a continuous backsheet web by continuously feeding first and second leg elastic members between first and second layers of backsheet material and securing the first and second leg elastic members between the first and second layers of backsheet material in first and second leg elastic anchoring portions arranged intermittently along corresponding first and second longitudinal edges of the continuous backsheet web and being separated by leg elastic release portions, the first and second leg elastic members being secured to the continuous backsheet web in the leg elastic anchoring portions while being held in a stretched state;
forming a continuous article web of interconnected hygienic articles, the continuous article web incorporating the continuous backsheet web;
severing individual hygienic articles from the continuous article web and simultaneously severing the first and second leg elastic members in the leg elastic release portions between the intermittently arranged leg elastic anchoring portions, causing the severed leg elastic elements to relax and retract within the leg elastic release portions.

24. The method according to claim 23, wherein forming the continuous article web includes joining one or more of the following components to the continuous backsheet web:
a topsheet;
an absorbent member;
a waistband component;
side panels;
an acquisition web;
fastener elements;
barrier cuffs;
a wetness indicator.

25. The hygienic article according to claim 1, wherein the first and second side flaps are attachment flaps and are secured to the hygienic article between the topsheet and the backsheet arrangement in respective flap anchoring areas.

26. The hygienic article according to claim 25, wherein a portion of the backsheet extends between the leg elastic members and the top sheet.

27. The hygienic article according to claim 25, wherein the leg elastic anchoring portions and the leg elastic members are arranged laterally completely outward of inner edges of the flap anchoring areas.

28. The method according to claim 23, wherein the first and second side flaps are attachment flaps and are secured to the hygienic article between the topsheet and the backsheet arrangement in respective flap anchoring areas.

29. The method according to claim 28, wherein a portion of the backsheet extends between the leg elastic members and the top sheet.

30. The method according to claim 28, wherein the leg elastic anchoring portions and the leg elastic members are arranged laterally completely outward of inner edges of the flap anchoring areas.

31. The hygienic article according to claim 1, wherein a portion of the backsheet arrangement extends between the leg elastic members and the top sheet.
